(19) United States
(12) Patent Application Publication
(10) Pub. No.: US 2022/0161635 A1
(43) Pub. Date: May 26, 2022

(54) Air Diffuser

(71) Applicant: Nikolay Reshetnyak, Moscow (RU)

(72) Inventor: Nikolay Reshetnyak, Moscow (RU)

(21) Appl. No.: 17/144,665

(22) Filed: Jan. 8, 2021

Related U.S. Application Data
(63) Continuation of application No. 29/759,296, filed on Nov. 20, 2020.

(51) Int. Cl. B60H 3/00; A61L 9/12

(52) U.S. Cl. CPC B60H 3/0028; A61L 2209/16; A61L 2209/15; A61L 9/12

(57) ABSTRACT

Disclosed is a system for diffusing oil. In one embodiment, the disclosed system features an essential oil or aromatic substance diffuser, aroma pads, and a convenient packaging.

### CROSS-REFERENCE TO RELATED APPLICATIONS

This application is a continuation of U.S. Design patent application Ser. No. 29/759,296 (filed Nov. 20, 2020) for an “air diffuser.” That document is incorporated by reference in its entirety.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of air diffusers and air conditioners.

### Background of the Invention

Aroma therapy is a treatment technique that diffuses essential oils or other aromatic substance into the air in the vicinity of a patient. Some experts believe the diffused substances engage receptors in the patient's nasal cavities to open the sinus and activate certain areas of the brain that release “feel-good” chemicals like serotonin. Aroma therapy has been used help patients relax, fall asleep, relieve stress, treat anxiety or to depression, kill certain bacteria, and alleviate certain types of pain.

Aroma therapy is often performed at a spa or medical clinic. However, spas and clinics are expensive or otherwise inaccessible to certain patients in need of aroma therapy. When affordable, spas and clinics are still not entirely satisfactory for aromatherapy because such spas and clinics typically require a scheduled appointment. These drawbacks mean that certain patients cannot regularly receive aroma therapy. Accordingly, there exists a need for an air diffuser for aroma therapy that is accessible for convenient on demand aroma therapy.

### SUMMARY OF THE INVENTION

In view of the foregoing, an object of this specification is to disclose a system for diffusing oil. In one embodiment, the disclosed system features an essential oil or aromatic substance diffuser, aroma pads, and a convenient packaging. In one example, the diffuser is a spherical platform configured to receive a replaceable aroma pad. The diffuser could suitably feature a clip which is used to attach the diffuser to a support. In this example, the aroma pads and diffuser are included in a single tubular package.

In one mode of operation, the disclosed system may be used to makeshift an automobile into an aromatherapy spa or clinic. In use, an essential oil is applied to the aroma pad which is inserted into the diffuser. To insert the aroma pad into the diffuser a rim of the diffuser is removed from the platform and the pad is slid onto the platform after an aromatic substance has been applied to the pad. To attach the diffuser to a vent, the clip is attached to a structure of the automobile. In one example, the structure is a vent grate. When the oil has been sufficiently evaporated or diffused from the pad, the pad may be removed from the diffuser and oil may be reapplied to the pad or the pad may be discarded. Continuing this example, the packaging is tubular and discloses instructions on how to use and set up the diffuser.

Advantages of the preferred embodiment include an air diffuser that does not require batteries or fire (like candles). An automobile can be converted into an aromatherapy spa by simply applying a few drops of an aromatic substance or essential oil to an aroma pad, inserting the pad into the diffuser and attaching the diffuser to a structure of the automobile.

### LISTING OF RELATED ART

The following is a list of related art:

U.S. D803377 to Gobber discloses a, “Air freshener.”

U.S. D820964 to Servaire discloses a, “Air freshener.”

U.S. D830530 to Webster discloses a, “Scent dispenser.”

U.S. D850600 to Gobber discloses a, “Air freshener having depressed front center circles and rear clip.”

U.S. D878539 to Lu discloses a, “Car diffuser clip.”

U.S. D464565 to Weinstein discloses, “Tubular packaging.”

U.S. D468636 to Weinstein discloses a, “Tubular packaging.”

U.S. D550829 to Buthier discloses a, “Air freshener device.”

GB2449747A to Mclaren discloses a, “Tubular packaging container and method for making a tubular packaging container.”

JP2005154012A to Spatafora discloses a, “Folding method and device for closing ends of tubular packaging material.”

JP2008532856A to Michaelski discloses a, “Packaging containers, especially cans.”

U.S. Pat. No. 5,794,842 to Hallam discloses, “Tubular packaging containers.”

U.S. Pat. No. 8,763,797 to Guillon discloses a, “Closed tubular container comprising a compensation device for the packaging of tablets.”

U.S. D385344 to Green discloses a, “Propeller air freshener.”

U.S. D532893 to Wu discloses a, “Air freshener device.”

U.S. D544088 to Wu discloses a, “Air freshener device.”

U.S. D549812S1 to Fellows discloses a, “4-chamber air freshener.”

U.S. D551333S1 to Buthier discloses a, “Air freshener device.”

U.S. D555778 to Fellows discloses a, “5-chamber air freshener.”

U.S. D629881 to Valentino discloses a, “Fragrance emanation apparatus.”

U.S. D715915 to Schouten discloses a, “Air freshener.”

U.S. D751683 to Furner discloses a, “Volatile dispenser.”

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is a perspective view of the air diffuser;

FIG. 2 is a front view of the air diffuser of FIG. 1;

FIG. 3 is a back view of the air diffuser of FIG. 1;

FIG. 4 is a left-side view of the air diffuser of FIG. 1;

FIG. 5 is a right-side view of the air diffuser of FIG. 1;

FIG. 6 is an exploded perspective view of the air diffuser of FIG. 1

FIG. 7 is an exploded left-side view of the air diffuser of FIG. 1;

FIG. 8 is an exploded right-side view of the air diffuser of FIG. 1 FIG. 9 is an exploded side view of the air diffuser of FIG. 1;

FIG. 9 is perspective view of an aroma pad;

FIG. 10 is a diagram of a preferred method of loading the aroma pad into a diffuser of FIG. 1; and,

FIG. 11 is an exploded top view of the air diffuser, a stack of aroma pads and packaging.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PROFFERED EMBODIMENTS

Disclosed is a system for diffusing oil. In one embodiment, the disclosed system features an essential oil or aromatic substance diffuser, aroma pads, and a convenient packaging. The more specific details of a preferred embodiment are disclosed in connection with the figures.

In one example, the diffuser is a spherical platform configured to receive a replaceable aroma pad. The diffuser could suitably feature a clip which is used to attach the diffuser to a support. In this example, the aroma pads and diffuser are included in a single tubular package.

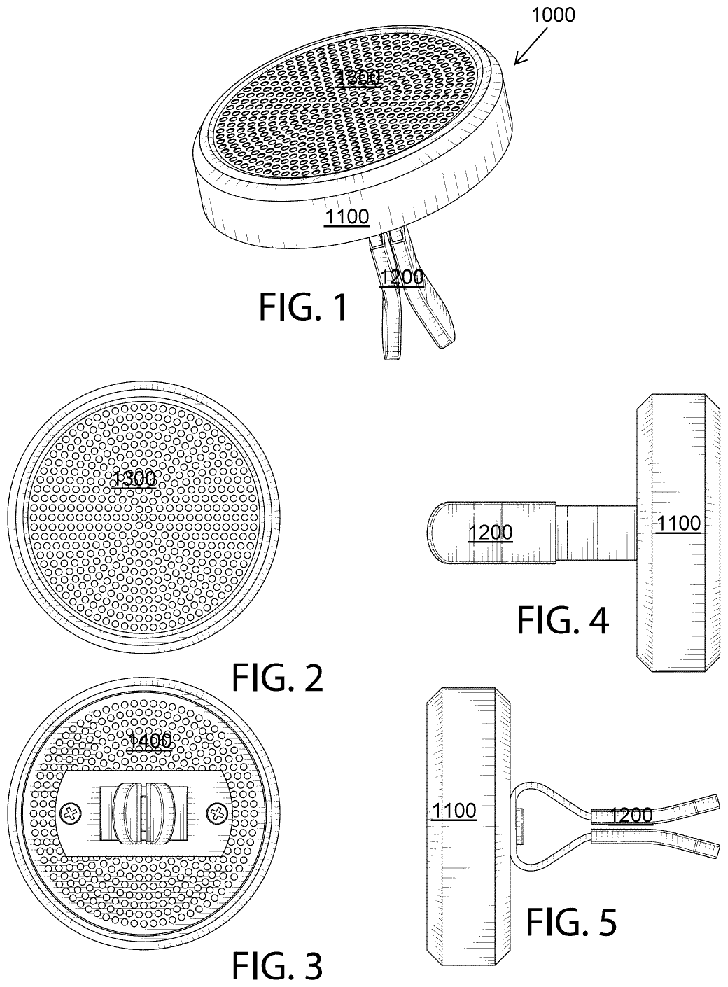

FIG. 1 is a perspective view of the air diffuser 1000. FIGS. 2 through 5 are respectively the front, back, left-side, and right-side view of the diffuser 1000. As shown, the diffuser 1000 appears to be a disc with a rim 1100 and a clip 1200. The diffuser has an exposed top surface 1300 with a plurality of air holes therethrough. The diffuser also has an exposed back surface 1400 with a plurality of airholes and a base for attaching thereto the clip 1200 so that it extends perpendicular to the bottom surface 1400.

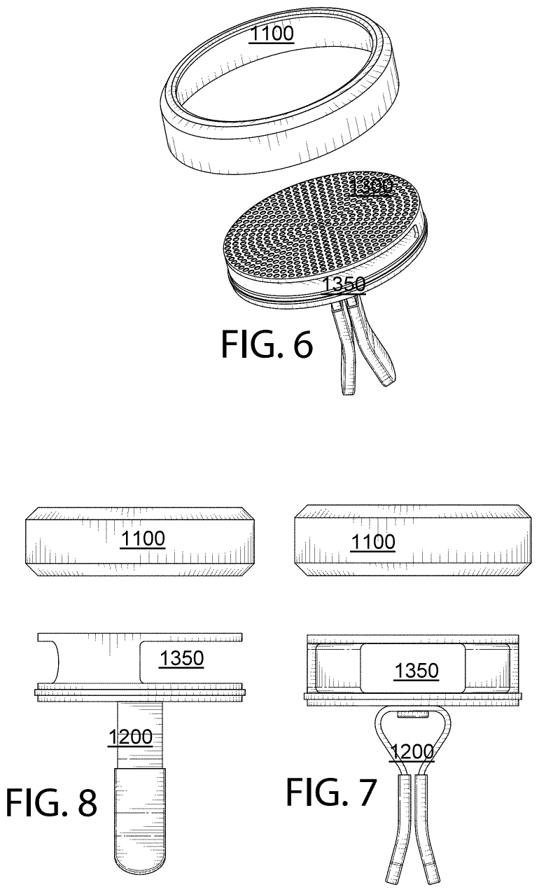

FIG. 6 is an exploded perspective view of the air diffuser 1000. FIGS. 7 and 8 are exploded side views of the air diffuser 1000. As shown, the rim 1100 of the diffuser is removable from the top and bottom surfaces 1300/1400 of the diffuser 1000. As shown, a platform 1350 is disposed between the two surfaces 1300/1400. Suitably, the rim may be snapped into position between the two surfaces to cover the platform 1350 or removed from between the two surfaces to expose the platform 1350.

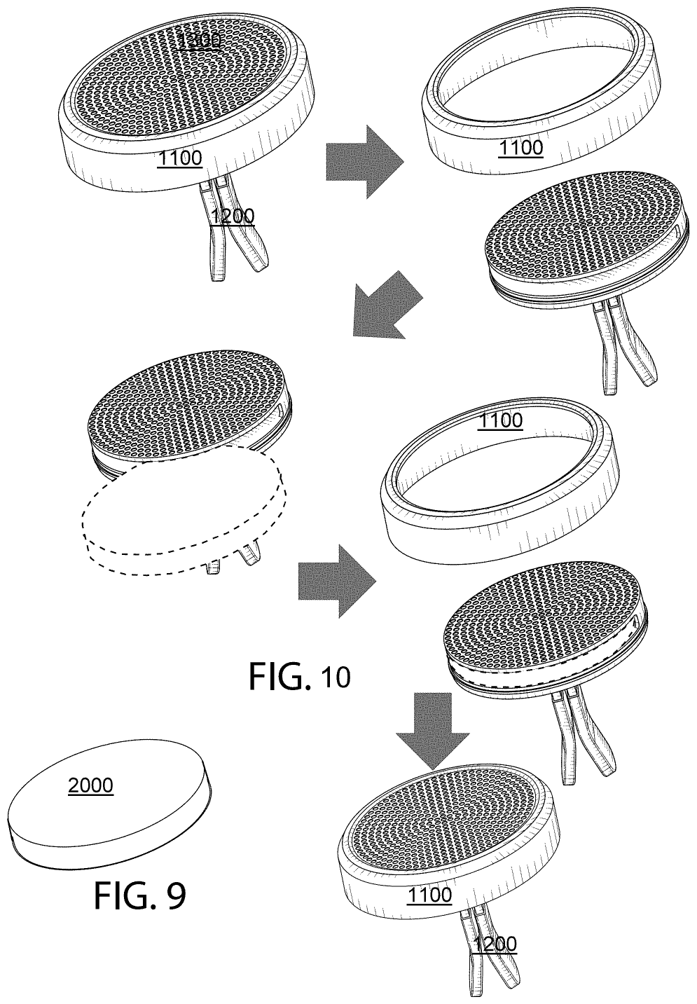

FIG. 9 is perspective view of an aroma pad 2000. As shown, the aroma pad is a semi-rigid disc of fabric or other fibrous material that can be inserted on the platform 1350. In one embodiment, the aroma pad 2000 is made of gauze or other mesh material and the like.

FIG. 10 is a diagram of a preferred method of loading the aroma pad into a diffuser of FIG. 1. In one mode of operation that is depicted in this figure, the disclosed system may be used to makeshift an automobile into an aromatherapy spa or clinic. In use, an essential oil is applied to the aroma pad 2000 which is inserted into the diffuser 1000. To insert the aroma pad 2000 into the diffuser 1000, a rim 1100 of the diffuser 1000 is removed from covering the platform 1350 and the pad 2000 is slid onto the platform 1350 after an aromatic substance (not shown) has been applied to the pad 2000. To attach the diffuser 100 to a vent or other structure of an automobile, the clip 1200 is spread is attached to a structure by placing the structure in between the clip 1200 arms. In one example, the structure is a vent grate of a car's air conditioning. When the oil has been sufficiently evaporated or diffused from the pad 2000, the pad may be removed from the diffuser and oil may be reapplied to the pad or the pad may be discarded.

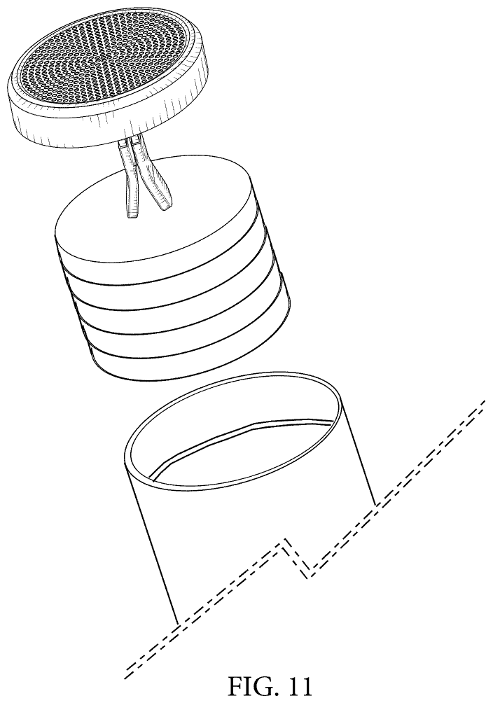

FIG. 11 is an exploded top view of the air diffuser, a stack of aroma pads and packaging. Continuing this example, the packaging is tubular and discloses instructions on how to use and set up the diffuser. There may be a plurality of aroma pads disposed in the packaging.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting to the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A system for diffusing oil comprising:
an essential oil or aromatic substance diffuser;
a plurality of aroma pads; and,
tubular packaging that holds the diffuser over a stack of the plurality of aroma pads.

2. The system of claim 1 wherein the diffuser features a spherical platform configured to receive one of the plurality aroma pads after the diffuser and pads have been removed from the packaging.

3. The system of claim 2 wherein the diffuser features a clip that attaches the diffuser to a support.

4. The system of claim 3 wherein the diffuser is a disc with a rim and the clip.

5. The system of claim 4 wherein the disc of the diffuser has an exposed top surface with a plurality of air holes therethrough.

6. The system of claim 5 wherein the disc of the diffuser also has an exposed back surface 1400 with a plurality of airholes and a base for attaching thereto the clip 1200 so that said clip extends perpendicular to the bottom surface of the disc.

7. The system of claim 6 where the rim of the diffuser is removable from the top and bottom surfaces and wherein the platform is disposed between the two surfaces.

8. The system of claim 7 wherein the rim is snapped into position between the two surfaces to cover the platform or removed from between the two surfaces to expose the platform.
